(19) United States
(12) Patent Application Publication
(10) Pub. No.: US 2025/0160844 A1
(43) Pub. Date: May 22, 2025

(54) STEERABLE LEFT ATRIAL APPENDAGE CLOSURE DEVICE

(71) Applicant: BOSTON SCIENTIFIC SCIMED, INC., MAPLE GROVE, MN (US)

(72) Inventors: James M. Anderson, Corcoran, MN (US); Jerry Wang, Edina, MN (US); Joshua Mark Inouye, Brooklyn Park, MN (US)

(73) Assignee: BOSTON SCIENTIFIC SCIMED, INC., MAPLE GROVE, MN (US)

(21) Appl. No.: 18/954,761

(22) Filed: Nov. 21, 2024

Related U.S. Application Data
(60) Provisional application No. 63/601,894, filed on Nov. 22, 2023.

(51) Int. Cl. A61B 17/12; A61B 17/00; A61B 90/00; A61M 25/01

(52) U.S. Cl. CPC A61B 17/12122; A61B 17/12172; A61B 2017/00323; A61B 2017/00597; A61B 2017/00623; A61B 2017/00632; A61B 2017/12054; A61B 2090/3966; A61M 25/0147

(57) ABSTRACT

Medical devices as wells as methods for making and using medical devices are disclosed. An example medical device may include a left atrial appendage device. The occlusive medical device includes an expandable frame configured to shift between a first configuration and an expanded configuration and a fabric disposed along at least a portion of the expandable frame. The occlusive medical device may cooperate with a delivery device to permit steering of the occlusive medical device.

### CROSS-REFERENCE TO RELATED APPLICATIONS

This application claims the benefit of priority of U.S. Provisional Application No. 63/601,894 filed Nov. 22, 2023, the entire disclosure of which is hereby incorporated by reference.

### TECHNICAL FIELD

The present disclosure pertains to medical devices, and methods for manufacturing medical devices. More particularly, the present disclosure pertains to occlusive devices such as those deployed adjacent to the left atrial appendage.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a medical assembly for treating a left atrial appendage (LAA). The medical assembly includes a delivery device adapted to deliver an occlusive implant to the LAA and an occlusive implant adapted to be delivered via the delivery device. The delivery device includes one or more steering wires that are adapted to releasably engage an occlusive implant and an engagement member movable between an engaged position in which the one or more steering wires are held engaged with the occlusive implant and a disengaged position in which the one or more steering wires are released from engagement with the occlusive implant. The occlusive implant includes an expandable frame configured to shift between a first configuration and an expanded configuration, an occlusive covering extending over at least part of the expandable frame, and a steering interface coupled to the expandable frame, the steering interface adapted to releasably engage the one or more steering wires when the engagement member is in the engaged position.

Alternatively or additionally, the steering interface may include an at least hemispherical interface.

Alternatively or additionally, the at least hemispherical interface may include an outer surface and one or more indentations formed within the outer surface, each of the one or more indentations adapted to releasably engage a distal end of one of the one or more steering wires.

Alternatively or additionally, the distal end of each of the one or more steering wires may include an enlarged diameter distal tip.

Alternatively or additionally, the delivery device may further include an outer shaft through which the one or more steering wires extend.

Alternatively or additionally, the delivery device may further include a deployment member extending through the outer shaft.

Alternatively or additionally, the engagement member may include a helical structure extending distally from a distal end of the deployment member.

Alternatively or additionally, the deployment member may include a midshaft extending through the outer shaft.

Alternatively or additionally, the engagement member may include a cylindrical coil extending distally from the deployment member.

Another example may be found in a medical assembly for treating a left atrial appendage (LAA). The medical assembly includes an occlusive implant and a delivery device adapted to deliver the occlusive implant to the LAA. The occlusive implant includes an expandable frame configured to shift between a first configuration and an expanded configuration, an occlusive covering extending over at least part of the expandable frame, and a spherical interface coupled to the expandable frame. The delivery device includes an elongate deployment mechanism having a distal anchor point, a basket secured to the distal anchor point of the elongate deployment mechanism, the basket movable between an engaged position in which the spherical interface is trapped within the basket and a disengaged position in which the spherical interface is released from the basket, and a sheath slidingly disposed relative to the basket, the sheath holding the basket in the engaged position when the sheath extends over the basket, the sheath adapted to be withdrawn proximally in order to allow the basket to regain the disengaged position.

Alternatively or additionally, the delivery device may be adapted to allow the occlusive implant to pivot passively relative to the delivery device.

Alternatively or additionally, the delivery device may be adapted to allow active steering at the distal anchor point.

Alternatively or additionally, the delivery device may be adapted to allow the occlusive implant to passively pivot relative to the distal anchor point and to also allow the distal anchor point to be steered.

Alternatively or additionally, the spherical interface may be adapted to be free to rotate within the basket when the basket is in the engaged position, thereby allowing the occlusive implant to pivot relative to the delivery device.

Alternatively or additionally, the delivery device may further include one or more steering wires that are adapted to engage the distal anchor point.

Alternatively or additionally, the one or more steering wires may be secured to the distal anchor point.

Another example may be found in a left atrial appendage closure (LAAC) device. The LAAC device includes an expandable frame configured to shift between a first configuration and an expanded configuration, an occlusive covering extending over at least part of the expandable frame, and a spherical interface coupled to the expandable frame, the spherical interface adapted to allow the LAAC device to pivot relative to a delivery device.

Alternatively or additionally, the spherical interface may include an outer surface and one or more indentations formed within the outer surface, each of the one or more indentations adapted to releasably engage each of one or more steering wires.

Alternatively or additionally, the spherical interface may include a radiopaque material.

Alternatively or additionally, the spherical interface may include titanium.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an illustrative left atrial appendage closure (LAAC) device extending through an example heart;

FIG. 2 is a schematic view of the illustrative LAAC device of FIG. 1, shown extending through another view of the example heart;

FIG. 3 is a perspective view of an illustrative LAAC device;

FIG. 4 is a perspective view of an illustrative LAAC device including a steering interface;

FIG. 5 is a perspective view of a portion of an illustrative steerable delivery device for delivering the illustrative LAAC device of FIG. 4, with the illustrative steerable delivery device shown in an engaged position;

FIG. 6 is a perspective view of a portion of the illustrative steerable delivery device of FIG. 5;

FIG. 7 is a perspective view of a portion of the illustrative steerable delivery device of FIG. 5, shown in a disengaged position;

FIG. 8 is a schematic side view of a portion of an illustrative steerable delivery device;

FIG. 9 is a schematic end view of the illustrative steerable delivery device of FIG. 8;

FIG. 10 is a schematic side view of the illustrative steerable delivery device of FIG. 8, showing inclusion of a helical structure forming an engagement member;

FIG. 11 is a schematic top view of the illustrative steerable delivery device of FIG. 10, shown disengaged from an illustrative LAAC device;

FIG. 12 is a schematic view of the illustrative steerable delivery device of FIGS. 8 through 11;

FIG. 13 is a schematic view of an illustrative steerable delivery device shown in a linear position;

FIG. 14 is a schematic view of the illustrative steerable delivery device of FIG. 13, shown in an articulated position;

FIG. 15 is a schematic view of the illustrative steerable delivery device of FIG. 13 in combination with an illustrative LAAC device, shown in a double articulated position;

FIG. 16 is a schematic view of an illustrative delivery device;

FIG. 17 is a schematic view of the illustrative delivery device of FIG. 16 in combination with an illustrative LAAC device, shown in an pivoted position;

FIG. 18 is a schematic view of a portion of an illustrative medical assembly including a retention wire;

FIG. 19 is a schematic view of a portion of an illustrative medical assembly including curved wires engaging a spherical interface on an illustrative LAAC device;

FIG. 20 is a schematic view of a portion of an illustrative medical assembly including angled wires engaging a spherical interface on an illustrative LAAC device;

FIG. 21 is a schematic view of an illustrative medical assembly shown in a linear configuration; and

FIG. 22 is a schematic view of the illustrative medical assembly of FIG. 21, shown in an articulated configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

The left atrial appendage (LAA) is a small sac attached to the left atrium of the heart as a pouch-like extension. In patients suffering from atrial fibrillation, the left atrial appendage may not properly contract with the left atrium, causing stagnant blood to pool within its interior, which can lead to the undesirable formation of thrombi within the left atrial appendage. Thrombi forming in the left atrial appendage may break loose from this area and enter the blood stream. Thrombi that migrate through the blood vessels may eventually plug a smaller vessel downstream and thereby contribute to stroke. Clinical studies have shown that the majority of blood clots in patients with atrial fibrillation are found in the left atrial appendage. As a treatment, medical devices have been developed which are positioned in the left atrial appendage and deployed to close off the ostium of the left atrial appendage. Over time, the exposed surface(s) spanning the ostium of the left atrial appendage becomes covered with tissue (a process called endothelization), effectively removing the left atrial appendage from the circulatory system and reducing or eliminating the number of thrombi which may enter the blood stream from the LAA. In an effort to reduce the occurrence of thrombi formation within the LAA and prevent thrombi from entering the blood stream from within the LAA, it may be desirable to develop medical devices and/or occlusive implants that close off the LAA from the heart and/or circulatory system, thereby lowering the risk of stroke due to thrombolytic material entering the blood stream from the LAA. Example medical devices and/or occlusive implants which seal the LAA (or other similar openings) are disclosed herein.

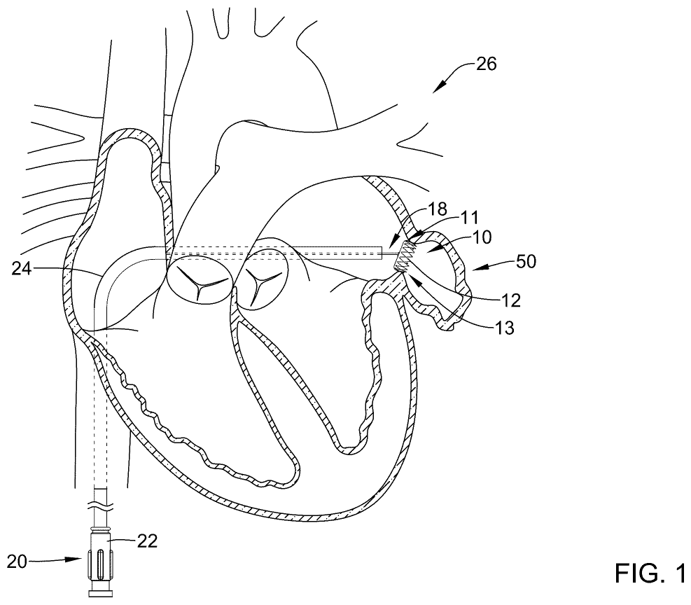

FIG. 1 illustrates an example occlusive implant 10 (e.g., a left atrial appendage medical device) positioned within the LAA 50. FIG. 1 further illustrates that the occlusive implant 10 may be inserted and advanced through a body lumen via an occlusive implant delivery system 20. In some instances, an occlusive implant delivery system 20 may include a delivery catheter 24 which is guided toward the left atrium via various chambers and lumens of the heart (e.g., the inferior vena cava, superior vena cava, the right atrium, etc.) to a position adjacent the LAA 50. The combination of the occlusive implant 10 temporarily secured relative to the occlusive implant delivery system 20 may be considered as being a medical assembly 26.

The delivery system 20 may include a hub 22. The hub 22 may be manipulated by a clinician to direct the distal end region of the delivery catheter 24 to a position adjacent the left atrial appendage 50. In some instances, an occlusive implant delivery system 20 may include a core wire 18. Further, a proximal end region 11 of the occlusive implant 10 may be configured to releasably attach, join, couple, engage, or otherwise connect to the distal end of the core wire 18. In some instances, the proximal end region 11 of the occlusive implant 10 may include a threaded insert coupled thereto. In some instances, the threaded insert may be configured to and/or adapted to couple with, join to, mate with, or otherwise engage a threaded member disposed at the distal end of a core wire 18. Other structures for releasably coupling and/or engaging the proximal end of the occlusive implant 10 to the distal end of the core wire 18 are also contemplated.

FIG. 1 further illustrates the occlusive implant 10 positioned adjacent the left atrial appendage 50 via the delivery catheter 24 (described above). It can be appreciated that in some examples, the occlusive implant 10 may be configured to shift between a first or collapsed configuration and a second or expanded configuration. For example, in some instances, the occlusive implant 10 may be in a collapsed configuration during delivery via the occlusive implant delivery system 20, whereby the occlusive implant 10 expands to an expanded configuration once deployed from the occlusion implant delivery system 20.

Additionally, FIG. 1 illustrates that the occlusive implant 10 may include an expandable frame or framework 12. The expandable framework 12 may be compliant and, therefore, substantially conform to and/or be in sealing engagement with the shape and/or geometry of a lateral wall of the LAA 50 in the expanded configuration. In some embodiments, the occlusive implant 10 may expand to a size, extent, or shape less than or different from a maximum unconstrained extent, as determined by the surrounding tissue and/or lateral wall of the LAA 50. Further, it can be appreciated that the elements of the expandable framework 12 may be tailored to increase the flexibility of the expandable framework 12 and/or the occlusive implant 10, thereby permitting the expandable framework 12 and/or the occlusive implant 10 to conform to the tissue around it, rather than forcing the tissue to conform to the expandable framework 12 and/or the occlusive implant 10. Additionally, in some instances, it may be desirable to design the occlusive implant 10 to include various features, components and/or configurations which improve the sealing capabilities of the occlusive implant 10 within the LAA.

FIG. 1 illustrates that the distal end region 13 of the expandable framework 12 may extend farther into the LAA 50 as compared to the proximal end region 11 of the expandable framework 12. It can be appreciated that as the expandable framework 12 is advanced into the LAA 50, the distal end region 13 may engage with tissue defining the left atrial appendage 50. In other words, in some examples the distal end region 13 may be considered the “leading” region of the expandable framework 12 as it enters into the LAA 50. However, this is not intended to be limiting. Rather, in some examples the proximal end region 11 may be considered the “leading” region of the expandable framework 12 as it enters into the LAA 50.

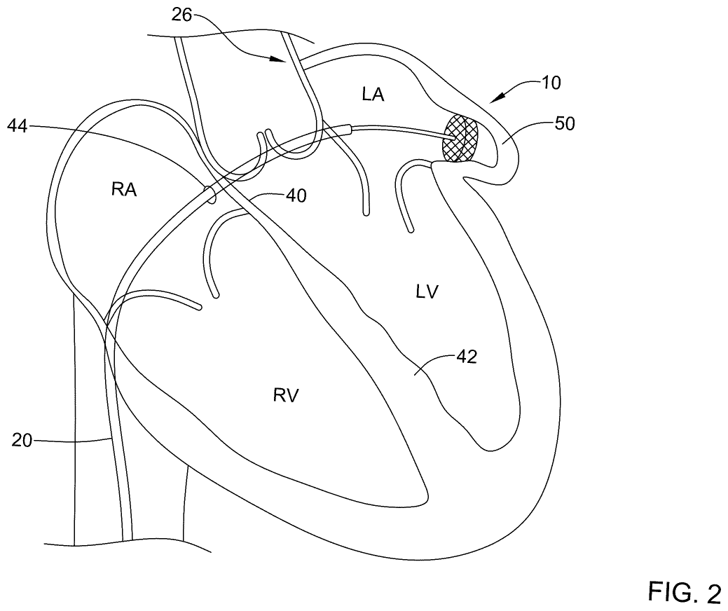

FIG. 2 provides another view of the occlusive implant 10 positioned within the LAA 50 via the occlusive implant delivery system 20. FIG. 2 provides a slightly different view of the interior of the heart, including the right atrium (RA), the left atrium (LA), the right ventricle (RV) and the left ventricle (LV). The heart includes an atrial septum 40 that separates the RA from the LA and a ventricular septum 42 that separates the RV from the LV. In some instances, the atrial septum 40 and the ventricular septum 42 may in combination simply be referred to as the septum, or sometimes may be referred to an atrioventricular septum. Regardless of terminology, the atrial septum 40 and the ventricular septum 42 separate the right side of the heart from the left side of the heart. In many instances, accessing the left side of the heart, whether it is the LA or the LV, is achieved by navigating through the vasculature into the right side of the heart, followed by puncturing the septum in order to reach the left side of the heart. For example, in order to reach the LV, the ventricular septum 42 may be breached in order to advance a device from the RV, through the opening formed in the ventricular septum 42, and into the LV. In order to reach the LA, the atrial septum 40 may be breached in order to advance a device from the RA, through the opening formed in the atrial septum 40, and into the LA. As shown, an opening 44 has been formed within the atrial septum 40, allowing the occlusive implant delivery system 20 to pass through the opening 44 and into the LA, and subsequently into the LAA 50.

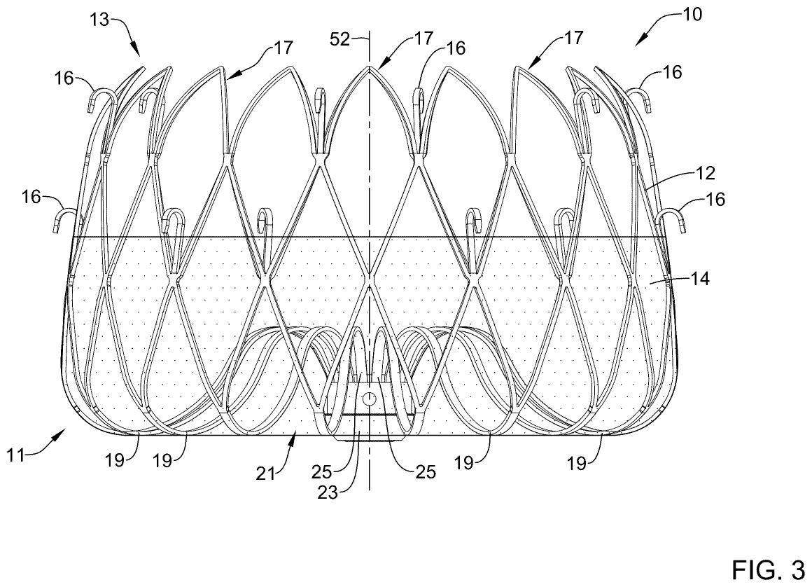

FIG. 3 illustrates an example occlusive implant 10. The occlusive implant 10 may include an expandable framework 12. The expandable framework 12 may include a proximal end region 11 and a distal end region 13. FIG. 2 further illustrates that the expandable framework 12 may include one or more projections 17 extending in a proximal-to-distal direction. In some instances (such as that shown in FIG. 3), plurality of projections 17 may extend circumferentially around a longitudinal axis 52 of the expandable framework 12. In other words, in some examples the projections 17 may resemble the peaks of a “crown” extending circumferentially around a longitudinal axis 52 of the expandable framework 12. While the above discussion (and the illustration shown in FIG. 3), shows a plurality of projections 17, it is contemplated that the occlusive implant 10 may include 1, 2, 3, 4, 5, 6, 7, 8, 9, 10, 11, 12 or more individual projections 17 disposed in a variety of arrangements along the expandable framework 12.

Additionally, FIG. 3 illustrates that the proximal end region 11 of the expandable framework 12 may include a plurality of support members 19 extending circumferentially around the longitudinal axis 52 of the expandable framework 12. FIG. 3 illustrates that that plurality of support members 19 may include one or more curved portions which are shaped such that they define a “recess” 21 extending distally into the expandable framework 12. As illustrated in FIG. 3, the recess 21 may extend circumferentially around the longitudinal axis 52. Further, FIG. 3 illustrates that each of the plurality of support members 19 may include a first end 25 which is attached to a central hub 23. It can be appreciated that the central hub 23 may be aligned along the longitudinal axis 52 of the expandable framework 12. As will be described in greater detail below, FIG. 3 illustrates that the hub 23 may be positioned such that it lies within the recess 21 defined by the plurality of support members 19.

The occlusive implant 10 may also include an occlusive member 14 disposed on, disposed over, disposed about, or covering at least a portion of the expandable framework 12. In some instances, the occlusive member 14 may be disposed on, disposed over, disposed about or cover at least a portion of an outer (or outwardly-facing) surface of the expandable framework 12. FIG. 3 further illustrates that the occlusive member 14 may extend only partially along the longitudinal extent of the expandable framework 12. However, this is not intended to be limiting. Rather, the occlusive member 14 may extend along the longitudinal extent of the expandable framework 12 to any degree (e.g., the full longitudinal extend of the expandable framework 12).

In some embodiments, the occlusive member 14 may be permeable or impermeable to blood and/or other fluids, such as water. In some embodiments, the occlusive member 14 may include a woven fabric/material or mesh, a non-woven fabric/material or mesh, a braided and/or knitted material, a fiber, a sheet-like material, a fabric, a mesh, a fabric mesh, a polymeric membrane, a metallic or polymeric mesh, a porous filter-like material, a covering, and/or other suitable construction. In some embodiments, the occlusive member 14 may prevent thrombi (i.e. blood clots, etc.) from passing through the occlusive member 14 and out of the left atrial appendage into the blood stream. In some embodiments, the occlusive member 14 may promote endothelialization after implantation, thereby effectively removing the left atrial appendage from the patient's circulatory system. Some suitable, but non-limiting, examples of materials for the occlusive member 14 are discussed below.

FIG. 3 further illustrates that the expandable framework 12 may include a plurality of anchor members 16 disposed about a periphery of the expandable framework 12. The plurality of anchor members 16 may extend radially outward from the expandable framework 12. In some embodiments, at least some of the plurality of anchor members 16 may each have and/or include a body portion and a tip portion projecting circumferentially therefrom, as shown in FIG. 2. Some suitable, but non-limiting, examples of materials for the expandable framework 12 and/or the plurality of anchor members 16 are discussed below.

In some examples, the expandable framework 12 and the plurality of anchor members 16 may be integrally formed and/or cut from a unitary member. In some embodiments, the expandable framework 12 and the plurality of anchor members 16 may be integrally formed and/or cut from a unitary tubular member and subsequently formed and/or heat set to a desired shape in the expanded configuration. In some embodiments, the expandable framework 12 and the plurality of anchor members 16 may be integrally formed and/or cut from a unitary flat member, and then rolled or formed into a tubular structure and subsequently formed and/or heat set to the desired shape in the expanded configuration. Some exemplary means and/or methods of making and/or forming the expandable framework 12 include laser cutting, machining, punching, stamping, electro discharge machining (EDM), chemical dissolution, etc. Other means and/or methods are also contemplated.

As illustrated in FIG. 3, the plurality of anchor members 16 disposed along the expandable framework 12 may include two rows of anchor members 16. However, this is not intended to be limiting. Rather, the expandable framework 12 may include a single row of anchor members 16. In other examples, the expandable framework 12 may include more than two rows of anchor members 16. For example, in some instances the expandable framework 12 may include 1, 2, 3, 4 or more rows of anchor members 16.

While FIG. 3 illustrates an expandable framework 12 which may be formed from a unitary member, this is not intended to be limiting. Rather, it is contemplated the expandable framework 12 may include a variety of different configurations which may be formed via a variety of manufacturing techniques.

As indicated above, the occlusive member 14 may include a woven fabric/material or mesh, a non-woven fabric/material or mesh, a braided and/or knitted material, a fiber, a sheet-like material, a fabric, a mesh, a fabric mesh, a polymeric membrane, a metallic or polymeric mesh, a porous filter-like material, a covering, and/or other suitable construction. The occlusive member 14 may be formed from a suitable material such as polyethylene terephthalate, polyester, nylon, acrylic materials, a polyolefin, and/or the like, combinations thereof, and/or other materials disclosed herein. In other instances, the occlusive material may include metallic mesh formed from nickel-titanium alloy, stainless steel, titanium, other materials disclosed herein, combinations thereof, and/or the like.

In some instances, the opening 44 formed within the atrial septum 42 may not be easily aligned with the LAA 50. In some instances, the exact position of the LAA 50 may vary from patient to patient, and thus there may not always be an optimal location for crossing the atrial septum 42. In some instances, other devices may be advanced from the RA, through the atrial septum 42, and into the LA before the occlusive implant delivery system 20 is advanced into the RA. As a result, the opening 44 that is formed within the atrial septum 42 may be positioned in accordance with the positional needs of another device, such as but not limited to a visualization device. Other devices may also be advanced through the opening 44 that is formed within the atrial septum 42 before the occlusive implant delivery system 20 is advanced. This may mean that the opening 44 is not appropriately aligned with the LAA 50. In some instances, the occlusive implant delivery system 20 itself may be advanced through a steerable catheter. In some instances, the occlusive implant delivery system 20 may itself be adapted to allow passive or even active steering of the occlusive implant 10 such that the occlusive implant 10 is able to better align within the LAA 50 regardless of where the opening 44 is formed within the atrial septum 42. In some instances, as will be discussed, the occlusive implant 10 may be able to passively pivot relative to the occlusive implant delivery system 20 as the occlusive implant 10 begins to enter and seat within the LAA 50. In some instances, as will be discussed, the occlusive implant delivery system 20 may include features that allow the occlusive implant 10 to be steered as the occlusive implant 10 crosses through the RA.

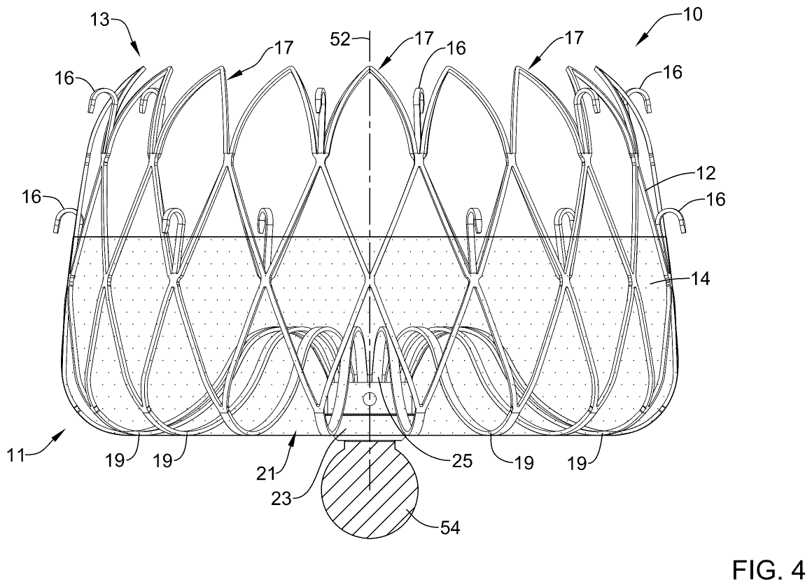

FIG. 4 shows the occlusive implant 10 including a steering interface 54. In some instances, the steering interface 54 may be secured to the expandable frame or framework 12. As an example, the steering interface 54 may be welded, soldered or adhesively secured to the expandable frame or framework 12. As another example, the steering interface 54 may be integrally formed as part of the expandable frame or framework 12. In some instances, the steering interface 54 may be adapted to releasably engage the core wire 18 in order to secure the occlusive implant 10 relative to the occlusive implant delivery system 20 until it is time to release the occlusive implant 10 from the occlusive implant delivery system 20.

In some instances, as will be discussed, the steering interface 54 may be adapted to engage a basket structure formed as part of the occlusive implant delivery system 20, and the steering interface 54 may be adapted to pivot relative to the basket structure. In some instances, as will be discussed, the steering interface 54 may include additional features that allow one or more steering wires to be releasably engaged with the steering interface 54. In some instances, the steering interface 54 may be considered as being spherical in shape. In some instances, the steering interface 54 may be considered as being hemispherical or even semispherical in shape. In some instances, the steering interface 54 may be made of, be coated with, or otherwise include a radiopaque material to render the steering interface 54 fluoroscopically visible. The steering interface 54 may be made of titanium, for example, or may be coated in gold or other radiopaque materials. In some instances, this may permit one or more radiopaque marker bands that might otherwise be disposed within the occlusive implant delivery system 20 to be excluded.

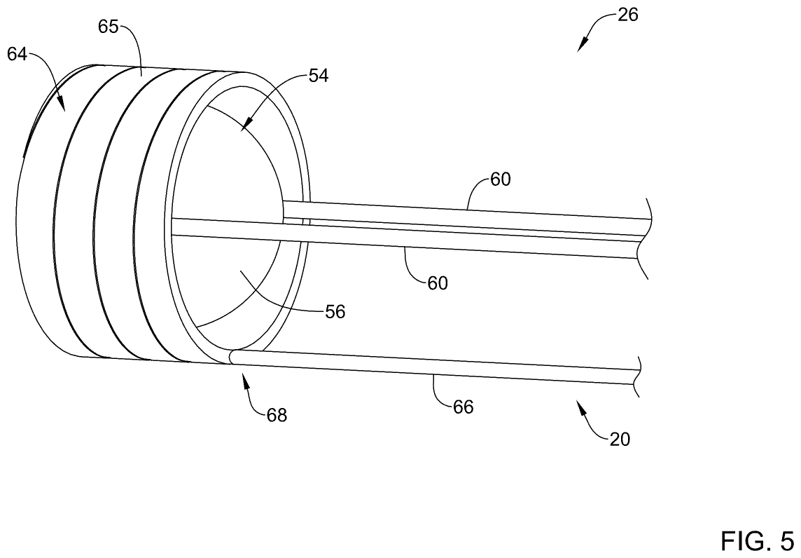

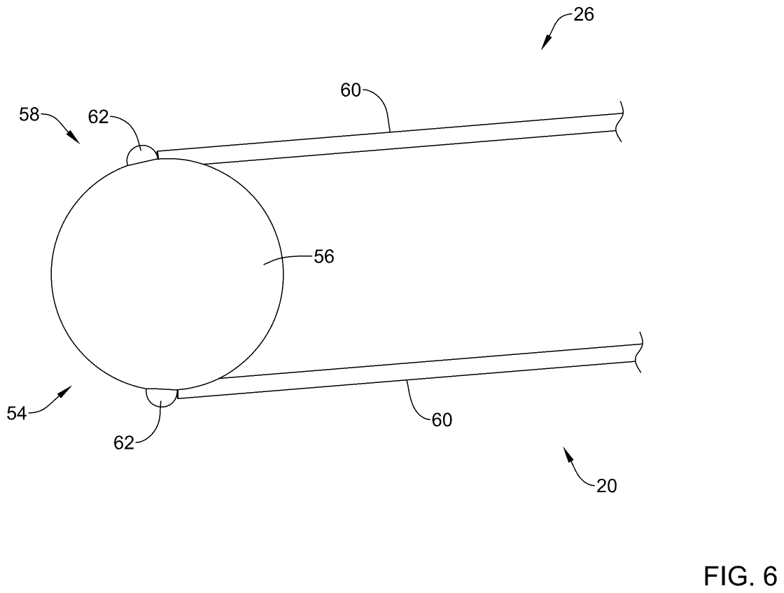

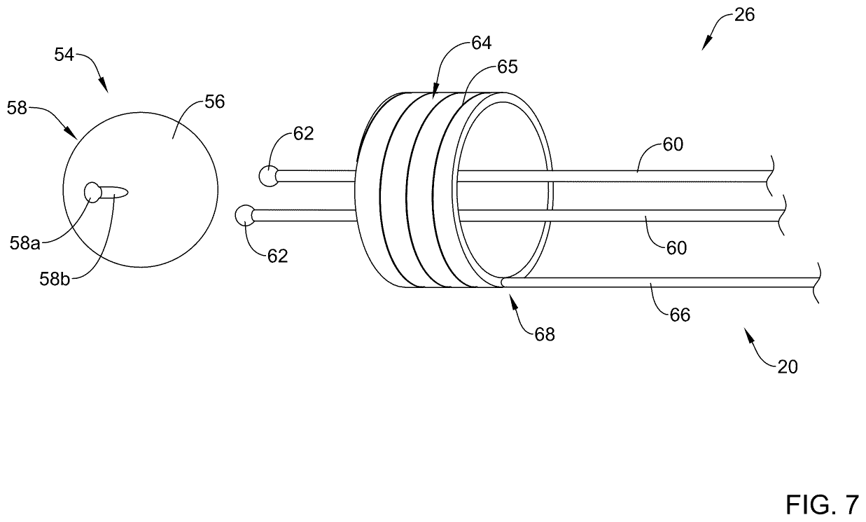

FIGS. 5, 6 and 7 are perspective views of a portion of an example of the medical assembly 26. While the medical assembly 26 includes the occlusive implant 10, in FIGS. 5, 6 and 7, the steering interface 54 is the only part of the occlusive implant 10 that is illustrated. The steering interface 54 includes an outer surface 56 that as seen in FIG. 7, includes one or more indentations 58 that are adapted to releasably accommodate steering wires 60 that are part of the occlusive implant delivery system 20. While referred to as steering wires 60, in some instances, the steering wires 60 may also function to releasably secure the occlusive implant 10 relative to the occlusive implant delivery system 20. In some instances, each of the indentations 58 may include a primary indentation 58a and a secondary indentation 58b. The primary indentation 58a may be adapted to releasably accommodate an enlarged diameter distal tip 62 of each of the steering wires 60. The secondary indentation 58b may be adapted to releasably accommodate the steering wire 60 itself when the enlarged diameter distal tip 62 is disposed within the primary indentation 58a.

An engagement member 64 may be movable between an engaged position (as shown in FIG. 5) in which the engagement member 64 keeps the steering wires 60 engaged with the corresponding indentations 58 formed within the outer surface 56 of the steering interface 54, and a disengaged position (as shown in FIG. 7) in which the steering wires 60 are released from engagement with the indentations 58 formed within the outer surface 56 of the steering interface 54. In this example, the engagement mechanism 64 may take the form of a cylindrical coil 65 that is adapted to fit over the steering adaptor 54 and is dimensioned to hold the enlarged diameter distal tips 62 of each of the steering wires 60 engaged with the indentations 58 when the engagement member 64 is positioned over the enlarged diameter distal tips 62 of each of the steering wires 60 and the indentations 58. In some instances, the occlusive implant delivery system 20 may include a deployment member 66. In some instances, the engagement member 64 is secured relative to a distal end 68 of the deployment member 66. In some instances, the engagement member 64 may be a distal portion of an elongate sheath (not shown) and the deployment member 66 may be a corresponding proximal portion of the elongate sheath.

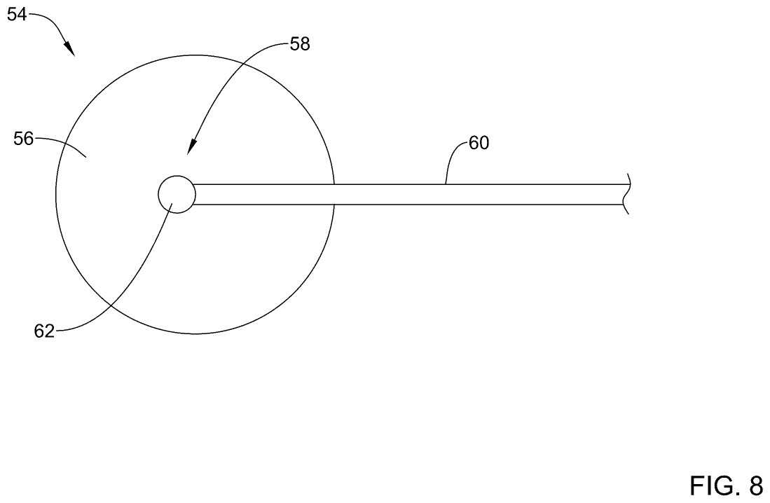

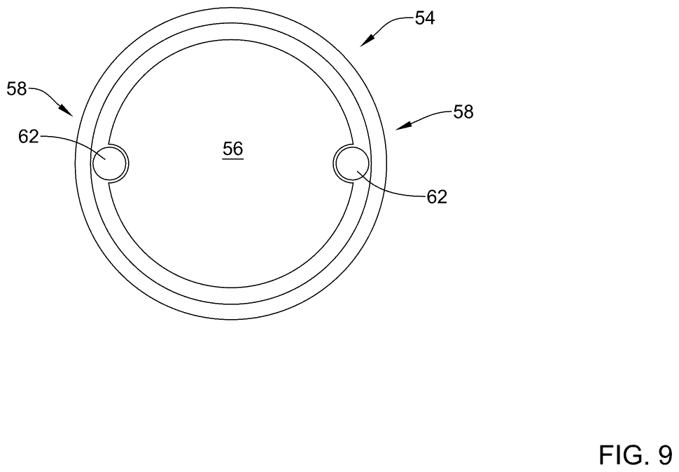

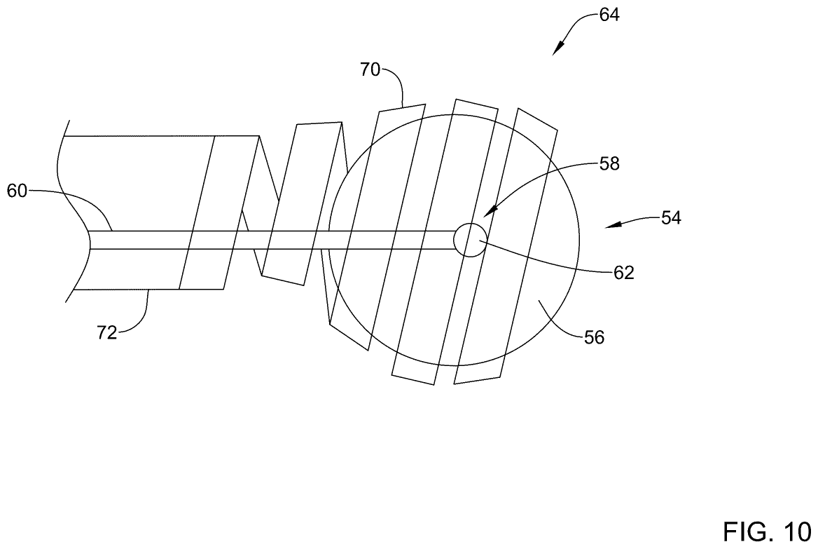

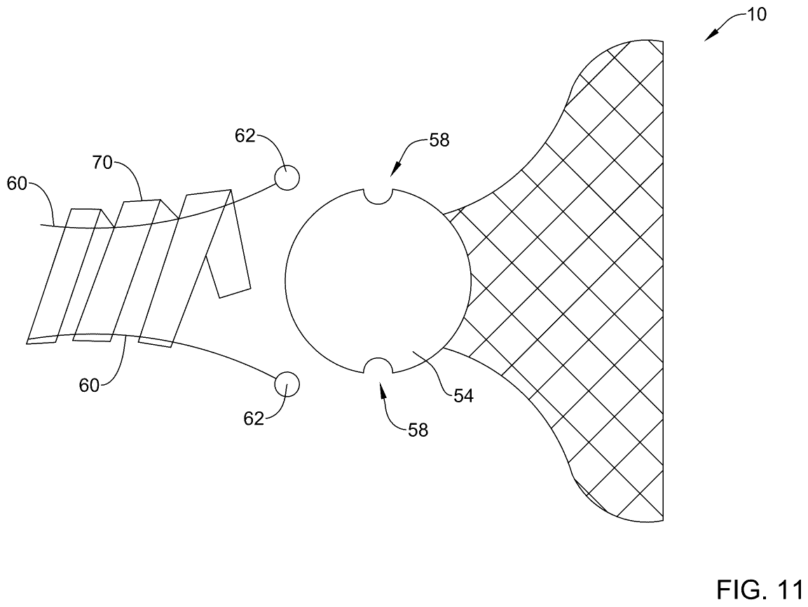

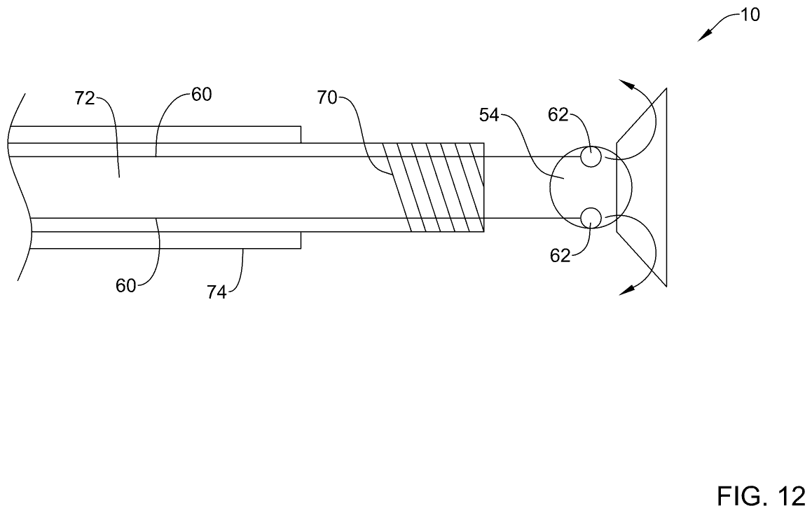

FIGS. 8, 9, 10 and 11 are schematic views of a portion of another example of the medical assembly 26. FIG. 8 is a side view, showing interaction between a steering wire 60 and the steering interface 54. FIG. 9 is a corresponding end view. FIG. 10 additionally shows the engagement member 64. FIG. 11 shows the steering wires 60 and the engagement member released from engagement with the steering interface 54. While the medical assembly 26 includes the occlusive implant 10, in FIGS. 8, 9 and 10, the steering interface 54 is the only part of the occlusive implant 10 that is illustrated. The steering interface 54 includes the outer surface 56 that includes one or more indentations 58 that are adapted to releasably accommodate the steering wires 60. While referred to as steering wires 60, in some instances, the steering wires 60 may also function to releasably secure the occlusive implant 10 relative to the occlusive implant delivery system 20. FIG. 12 shows the addition of a catheter shaft 74 through which the elongate shaft 72 and the steering wires 60 extend. It will be appreciated that the version of the medical assembly 26 shown in FIGS. 7 through 7 may similarly be disposed within the elongate shaft 72.

In some instances, the engagement member 64 may take the form of a helical retainer 70 that is secured to a distal end of an elongate shaft 72. The elongate shaft 72 may be considered as being a deployment member that may be advanced distally in order to advance the occlusive implant 10 into position within the LAA 50, prior to releasing the occlusive implant 10. In some instances, the helical retainer 70 may be withdrawn proximally from engagement with the steering interface 54 by simply withdrawing the elongate shaft 72 proximally. In some instances, the helical retainer 70 may be withdrawn proximally by rotating the elongate shaft 72 in an appropriate direction so that the helical retainer 70 withdraws proximally in almost a threaded manner

An engagement mechanism 64 may be movable between an engaged position (as shown in FIG. 5) in which the engagement mechanism 64 keeps the steering wires 60 engaged with the corresponding indentations 58 formed within the outer surface 56 of the steering interface 54, and a disengaged position (as shown in FIG. 7) in which the steering wires 60 are released from engagement with the indentations 58 formed within the outer surface 56 of the steering interface 54. In this example, the engagement mechanism 64 may take the form of a cylindrical coil that is adapted to fit over the steering adaptor 54 and is dimensioned to hold the enlarged diameter distal tips 62 of each of the steering wires 60 engaged with the indentations 58 when the engagement mechanism 64 is positioned over the enlarged diameter distal tips 62 of each of the steering wires 60 and the indentations 58. In some instances, the occlusive implant delivery system 20 may include a deployment member 66. In some instances, the engagement mechanism 64 is secured relative to a distal end 68 of the deployment member 66. In some instances, the engagement mechanism 64 may be a distal portion of an elongate sheath (not shown) and the deployment member 66 may be a corresponding proximal portion of the elongate sheath.

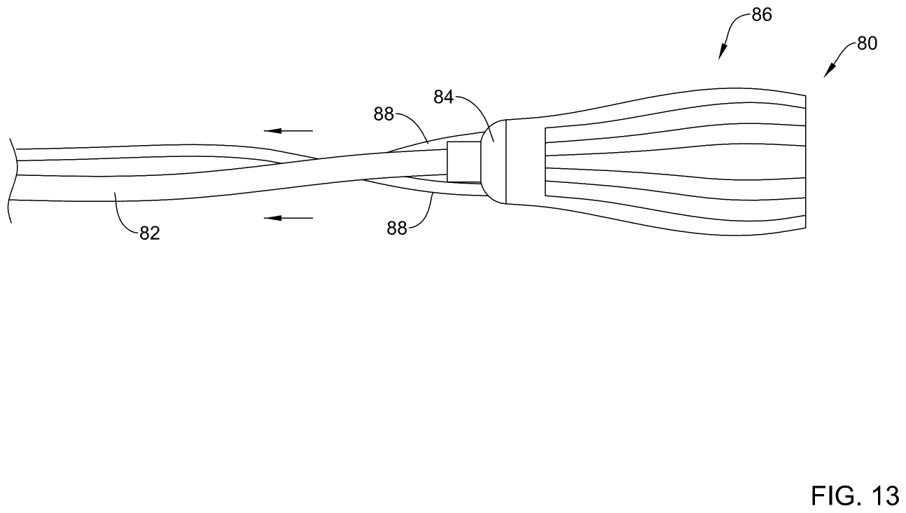

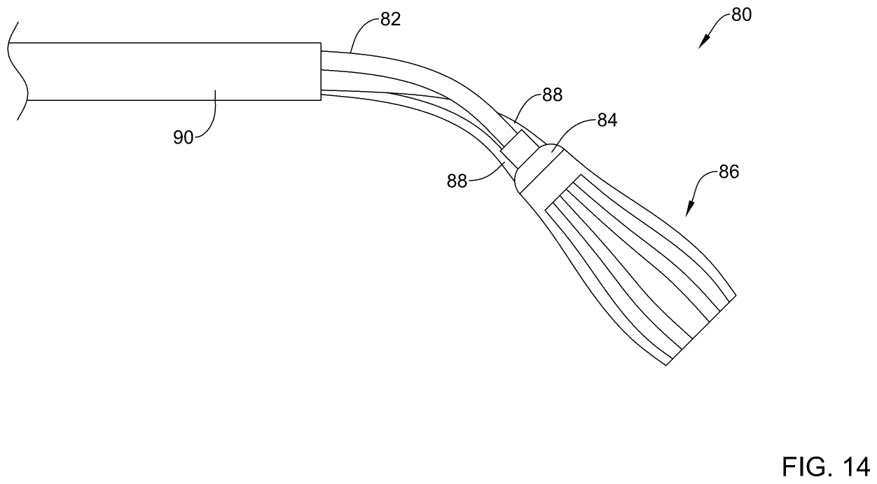

FIGS. 13 and 14 are schematic views of an illustrative delivery device 80 that may be used in delivering the occlusive implant 10. The illustrative delivery device 80 includes an elongate deployment member 82 that may be considered as being an example of the core wire 18 (FIG. 1). The elongate deployment member 82 includes a distal anchor point 84 formed in place or otherwise secured to a distal end of the elongate deployment member 82. A basket 86 extends distally from the distal anchor point 84. In some instances, the basket 86 may be adapted to accommodate the steering interface 54 within the basket 86 such that the steering interface 54 is able to pivot relative to the basket 86. In some instances, this relative movement is independent of any steering that is exerted. The delivery device 80 includes, as shown, two steering wires 88 that are secured to the distal anchor point 84 and extend proximally therefrom. As seen in FIG. 14, which shows the elongate deployment member 82 and the steering wires 88 extending distally out of a sheath 90, selecting pulling or pushing on one or both of the steering wires 88 can cause the elongate deployment member 82 to steer in a particular direction.

In some instances, the basket 86 may be adapted to allow the steering interface 54 to remain rotatably captive within the basket 86 such that translation of the basket 86 in a proximal direction or in a distal direction results in a corresponding movement of the occlusive implant 10 in the same proximal direction or distal direction. In some instances, the basket 86 may be adapted such that gentle movement of the basket 86 relative to the steering interface 54 allows the steering interface 54 to remain rotatably captive within the basket 86 while stronger movements of the basket 86 can cause the steering interface 54 to move free of the basket 86. In some instances, a movable sheath may be disposed over the basket 86 to keep the basket 86 engaged with the steering interface 54, and the movable sheath may be withdrawn from the basket 86 to allow the basket 86 to open and release the steering interface 54.

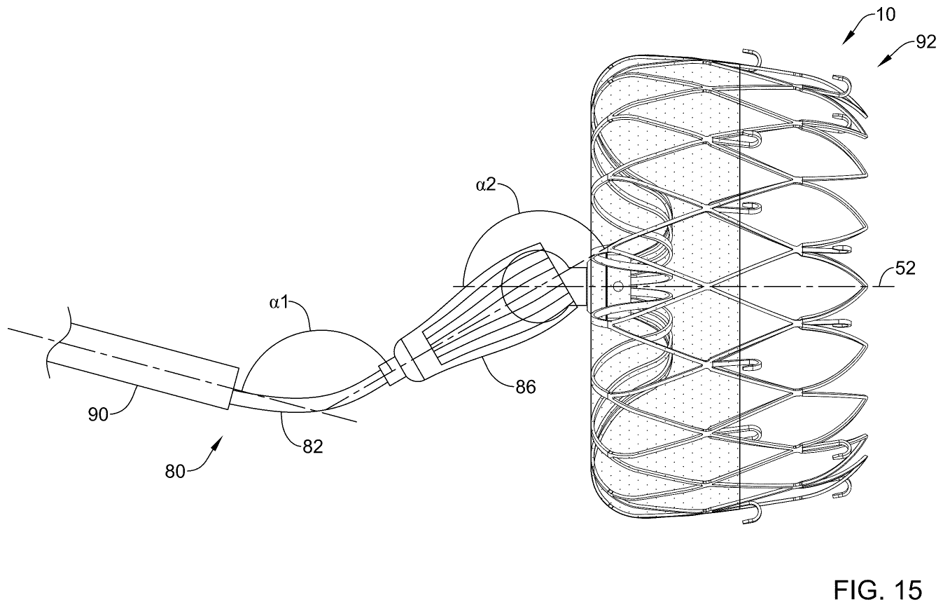

FIG. 15 is a schematic view of an illustrative medical assembly 92, which may be considered as another example of the medical assembly 26. The illustrative medical assembly 92 includes the occlusive implant 10 coupled to the delivery device 80 by virtue of the steering interface 54 being disposed within the basket 86. As can be seen, the medical assembly 92 allows multiple positioning or angling of the occlusive implant 10 relative to the delivery device 80. As indicated via the dashed lines, a first angle α1 is formed between the sheath 90 and the elongate deployment member 82 and a second angle α2 is formed between the elongate deployment member 82 and the longitudinal axis 52 of the expandable framework 12. By varying both angles, it is possible to achieve a greater flexibility in being able to align the occlusive implant 10 with the LAA 50. In some instances, the first angle α1 may be adjusted using the steering wires 88. In some instances, the second angle α2 may passively change as the occlusive implant 10 begins to impact and interact with the LAA 50.

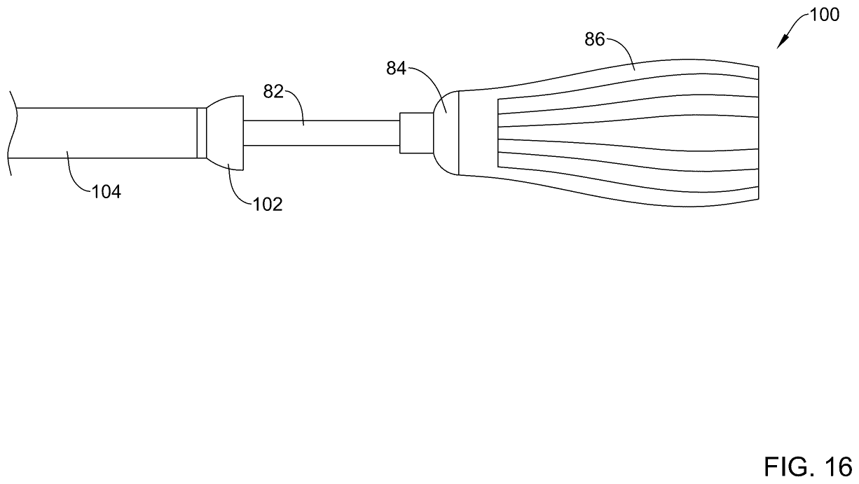

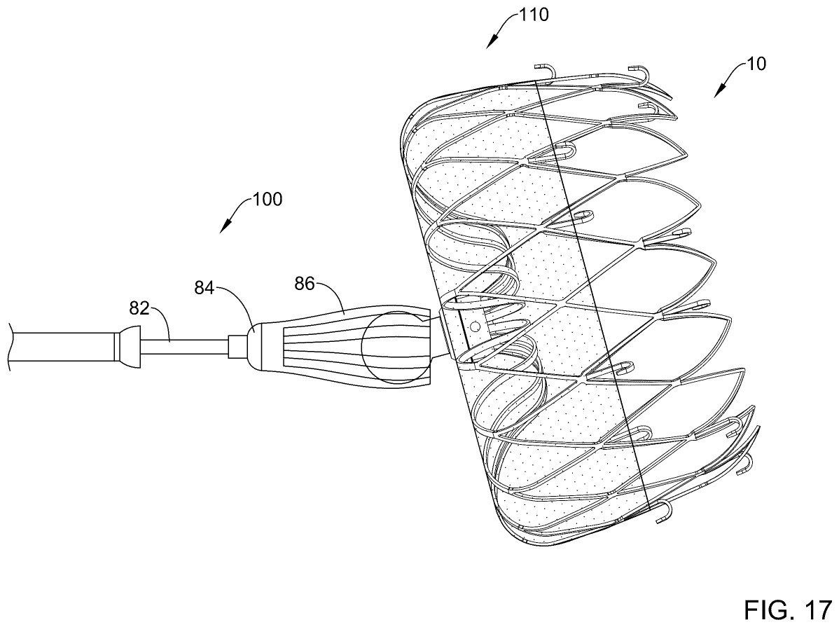

FIG. 16 is a schematic view of an illustrative delivery device 100 that may be used in delivering the occlusive implant 10. The delivery device 100 includes elongate deployment member 82 that may be considered as being an example of the core wire 18 (FIG. 1). The elongate deployment member 82 includes the distal anchor point 84 formed in place or otherwise secured to a distal end of the elongate deployment member 82. The basket 86 extends distally from the distal anchor point 84. In some instances, the basket 86 may be adapted to accommodate the steering interface 54 within the basket 86 such that the steering interface 54 is able to pivot relative to the basket 86. The delivery device 100 includes a sheath 102 that is adapted to accommodate the occlusive implant 10 and a delivery shaft 104. FIG. 17 is a schematic view of an illustrative medical assembly 110 that may be considered as being an example of the medical assembly 26. The illustrative medical assembly 110 includes the occlusive implant 10 secured relative to the delivery device 100 via an interaction between the steering interface 54 and the basket 86. Because the steering interface 54 is free to pivot within the basket 86, the occlusive implant 10 is free to pivot relative to the delivery device 100 as the occlusive implant 10 begins to engage the LAA 50.

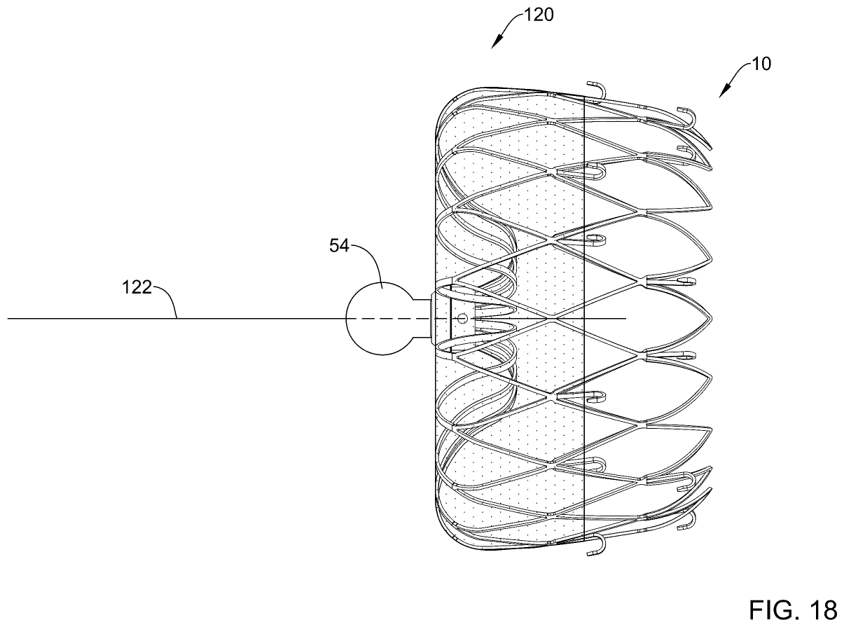

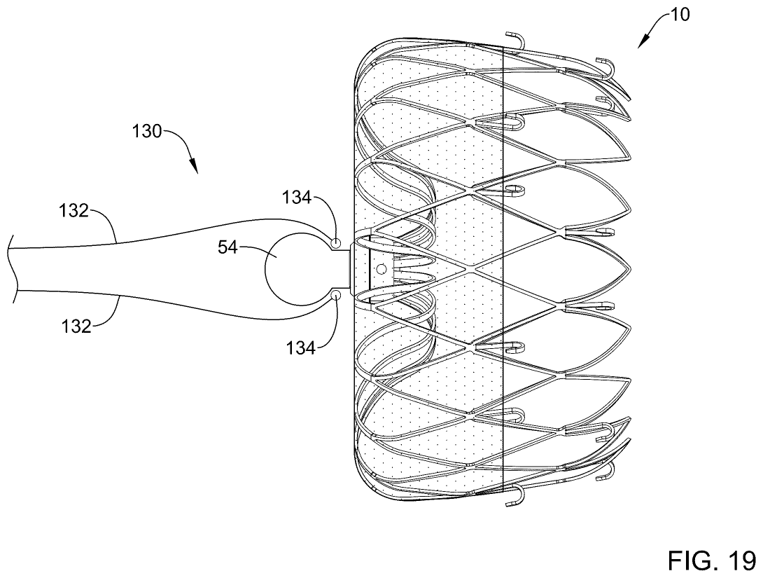

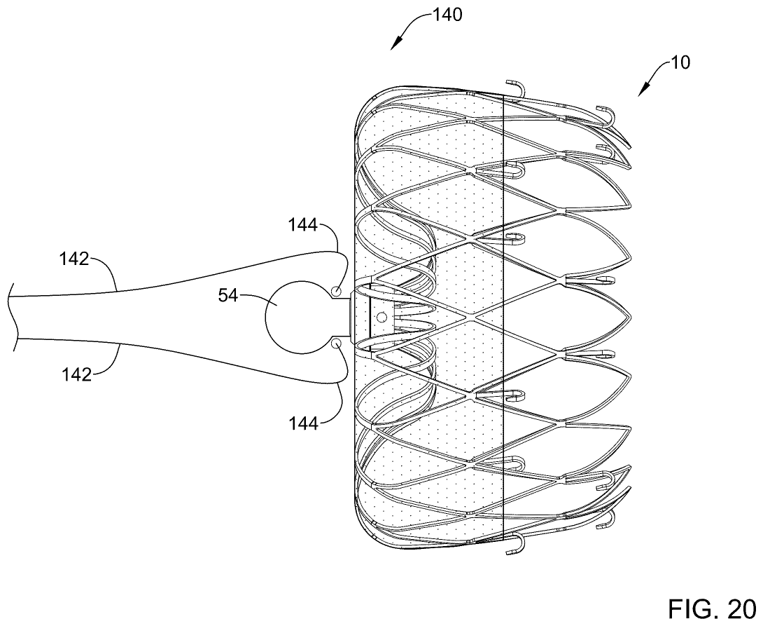

FIG. 18 is a schematic view of a portion of an illustrative medical assembly 120 that includes a retention wire 122 extending through the steering interface 54 to engage with the frame 12 of the occlusive implant 10. In some instances, the retention wire 122 provides improved retention control over the occlusive implant 10. The retention wire 122 may be combined with any of the delivery devices described herein. FIG. 19 is a schematic view of a portion of an illustrative assembly 130 that includes wires 132 that wrap around the steering interface 54 and include ball tips 134 at distal ends of each of the wires 132. The wires 132 may be used for retention, for example, and to allow the steering assembly 54 (and hence the occlusive implant 10) to freely pivot. FIG. 20 is a schematic view of a portion of an illustrative assembly 140 that includes wires 142 that wrap around the steering interface 54 and include angled prongs 144 at distal ends of each of the wires 142. The wires 142 may be used for retention, for example, and to allow the steering assembly 54 (and hence the occlusive implant 10) to freely pivot.

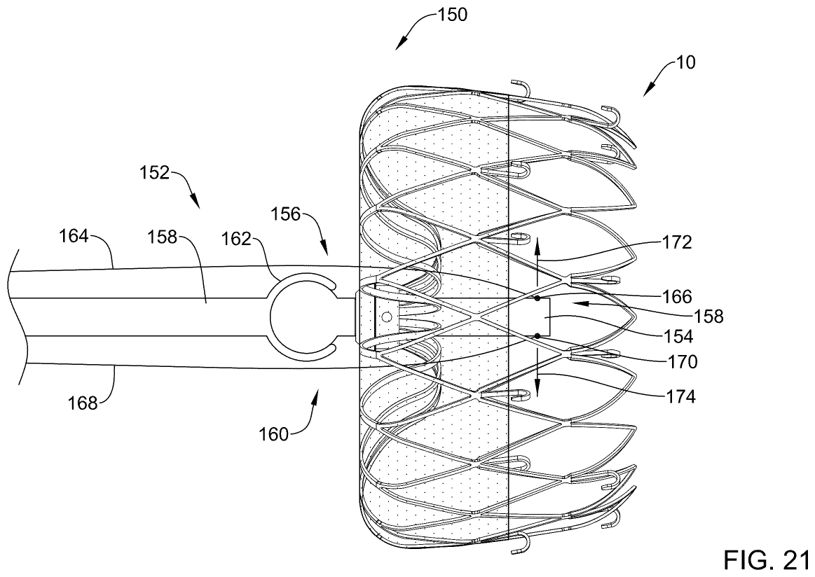

FIG. 21 is a schematic view of a portion of an illustrative medical assembly 150 showing the occlusive implant 10 secured relative to a delivery device 152. In some instances, as shown, the occlusive implant 10 includes a central rod 154 that extends into the occlusive implant 10. The central rod 154 may be secured relative to the expandable frame or framework 12 (not visible in this view), for example. The central rod 154 extends from a proximal end 156 to a distal end 158. In some instances, the distal end 158 is secured to the expandable frame or framework 12. In some instances, the central rod 154 may be additionally secured relative to the expandable frame or framework 12 at two or more points along the central rod 154, intermediate the proximal end 156 and the distal end 158. The proximal end 156 of the central rod 154 has a bulbous shape that may be at least hemispherical and thus functions in a manner similar to that of the steering interface 54 shown in previous drawings.

The delivery device 152 includes a central core member 158 that extends proximally from a distal end 160. The distal end 160 of the central core member 158 includes a socket 162 that rotatably engages the bulbous proximal end 156 of the central rod 154. In some instances, once the occlusive implant 10 is implanted within the LAA 50, the central core member 158 may be sharply withdrawn proximally in order to free the bulbous proximal end 156 of the central rod 154 from the distal end 160, leaving the central rod 154 secured relative to the occlusive implant 10. In some instances, the central rod 154 may remain secured to the central core member 158, and the central rod 154 may itself disengage from the expandable frame or framework 12.

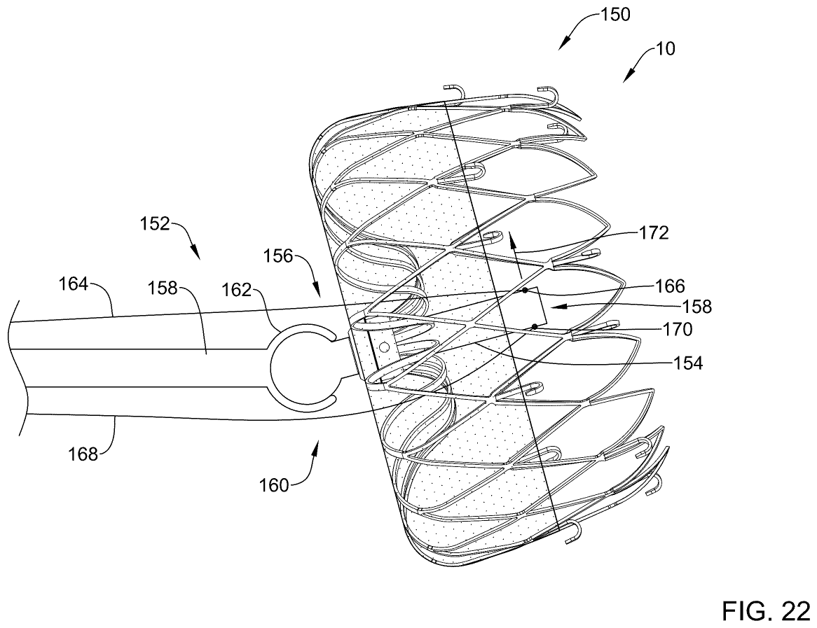

The delivery device 152 includes a first pull wire 164 having a distal end 166 secured to a first side of the central rod 154 and a second pull wire 168 having a distal end 170 secured to a second side of the central rod 154. Pulling on the first pull wire 164 may cause the central rod 154 (and hence the occlusive implant 10) to pivot in a first direction indicated by an arrow 172. Pulling on the second pull wire 168 may cause the central rod 154 (and hence the occlusive implant 10) to pivot in a second direction indicated by an arrow 174. Accordingly, it can be seen that the occlusive implant 10 can be steered. FIG. 21 shows the occlusive implant 10 in a linear configuration in which the occlusive implant 10 has not been steered or otherwise pivoted. FIG. 22 shows the occlusive implant 10 pivoted in the direction indicated by the arrow 172, as a result of the first pull wire 164 having been pulled proximally. Alternatively, this position of the occlusive implant 10 may also be achieved by pushing the second pull wire 168 in a distal direction, should the second pull wire 168 be stiff enough to accomplish this.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical assembly for treating a left atrial appendage (LAA), the medical assembly comprising:
a delivery device adapted to deliver an occlusive implant to the LAA, the delivery device including:
one or more steering wires adapted to releasably engage an occlusive implant;
an engagement member movable between an engaged position in which the one or more steering wires are held engaged with the occlusive implant and a disengaged position in which the one or more steering wires are released from engagement with the occlusive implant;
an occlusive implant adapted to be delivered via the delivery device, the occlusive implant including:
an expandable frame configured to shift between a first configuration and an expanded configuration;
an occlusive covering extending over at least part of the expandable frame; and
a steering interface coupled to the expandable frame, the steering interface adapted to releasably engage the one or more steering wires when the engagement member is in the engaged position.

2. The medical assembly of claim 1, wherein the steering interface comprises an at least hemispherical interface.

3. The medical assembly of claim 1, wherein the at least hemispherical interface comprises an outer surface and one or more indentations formed within the outer surface, each of the one or more indentations adapted to releasably engage a distal end of one of the one or more steering wires.

4. The medical assembly of claim 2, wherein the distal end of each of the one or more steering wires comprise an enlarged diameter distal tip.

5. The medical assembly of claim 1, wherein the delivery device further comprises an outer shaft through which the one or more steering wires extend.

6. The medical assembly of claim 5, wherein the delivery device further comprises a deployment member extending through the outer shaft.

7. The medical assembly of claim 6, wherein the engagement member comprises a helical structure extending distally from a distal end of the deployment member.

8. The medical assembly of claim 6, wherein the deployment member comprise a midshaft extending through the outer shaft.

9. The medical assembly of claim 6, wherein the engagement member comprises a cylindrical coil extending distally from the deployment member.

10. A medical assembly for treating a left atrial appendage (LAA), the medical assembly comprising:
an occlusive implant, the occlusive implant including:
an expandable frame configured to shift between a first configuration and an expanded configuration;
an occlusive covering extending over at least part of the expandable frame; and
a spherical interface coupled to the expandable frame; and
a delivery device adapted to deliver the occlusive implant to the LAA, the delivery device including:
an elongate deployment mechanism having a distal anchor point;
a basket secured to the distal anchor point of the elongate deployment mechanism, the basket movable between an engaged position in which the spherical interface is trapped within the basket and a disengaged position in which the spherical interface is released from the basket; and
a sheath slidingly disposed relative to the basket, the sheath holding the basket in the engaged position when the sheath extends over the basket, the sheath adapted to be withdrawn proximally in order to allow the basket to regain the disengaged position.

11. The medical assembly of claim 10, wherein the delivery device is adapted to allow the occlusive implant to pivot passively relative to the delivery device.

12. The medical assembly of claim 10, wherein the delivery device is adapted to allow active steering at the distal anchor point.

13. The medical assembly of claim 10, wherein the delivery device is adapted to allow the occlusive implant to passively pivot relative to the distal anchor point and to also allow the distal anchor point to be steered.

14. The medical assembly of claim 10, wherein the spherical interface is adapted to be free to rotate within the basket when the basket is in the engaged position, thereby allowing the occlusive implant to pivot relative to the delivery device.

15. The medical assembly of claim 10, wherein the delivery device further comprises one or more steering wires that are adapted to engage the distal anchor point.

16. The medical assembly of claim 10, wherein the one or more steering wires are secured to the distal anchor point.

17. A left atrial appendage closure (LAAC) device, comprising:
an expandable frame configured to shift between a first configuration and an expanded configuration;
an occlusive covering extending over at least part of the expandable frame; and
a spherical interface coupled to the expandable frame, the spherical interface adapted to allow the LAAC device to pivot relative to a delivery device.

18. The LAAC device of claim 17, wherein the spherical interface comprises an outer surface and one or more indentations formed within the outer surface, each of the one or more indentations adapted to releasably engage each of one or more steering wires.

19. The LAAC device of claim 17, wherein the spherical interface comprises a radiopaque material.

20. The LAAC device of claim 17, wherein the spherical interface comprises titanium.
